(19) United States
(12) Patent Application Publication
(10) Pub. No.: US 2010/0227900 A1
(43) Pub. Date: Sep. 9, 2010

(54) Use of Sulphur-Containing Heteroaromatic Acid Analogues as Bactericides

(75) Inventors: Lutz Aßmann, Langenfeld (DE); Karl-Wilhelm Münks, Meerbusch (DE); Peter Howard Davies, Muntinlupa (DE); Rolf Christian Becker, Burscheid (DE); Ingo Wetcholowsky, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 12/699,246

(22) Filed: Feb. 3, 2010

(30) Foreign Application Priority Data
Feb. 3, 2009 (EP) 09151904.1

(51) Int. Cl. A01N 43/828; C07D 285/06; A01P 15/00; C07D 275/03; A01N 43/80

(52) U.S. Cl. 514/361; 548/127; 548/214; 514/372

(57) ABSTRACT

The present invention relates to the use of compounds according to formula (I)

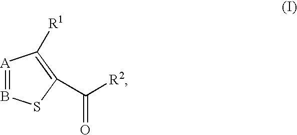

for controlling bacterial harmful organisms in useful plants.

Moreover, the present invention relates to a method for controlling bacterial harmful organisms in useful plants by treating them with compounds according to formula (I).

The present invention relates to the use of sulphur-containing heteroaromatic acid analogues according to formula (I)

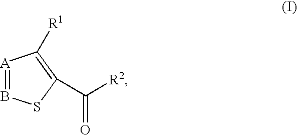

where
A is nitrogen or C-Hal,
B is nitrogen or C-Hal,
R¹ is hydrogen, halogen, cyano, C₁-C₆-alkyl, C₂-C₆-alkenyl, C₃-C₆-cycloalkyl, phenyl, C₁-C₆-alkyl which is substituted by Hal or cyano, C₂-C₆-alkenyl which is substituted by Hal or cyano, C₃-C₆-cycloalkyl which is substituted by Hal or cyano, or phenyl which is substituted by cyano, halogen, alkoxy,
R² is hydroxyl, C₁-C₆-thioalkyl, C₁-C₆-aminoalkyl, C₁-C₆-alkoxy, aniline, phenoxy,
or is C₁-C₆-alkoxy which is substituted by cyano, halogen, C₁-C₆-alkyl, C₁-C₆-alkoxy and/or C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl or is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is halogen,
   for controlling bacterial harmful organisms in useful plants.

Furthermore, the present invention relates to a method of controlling bacterial harmful organisms in useful plants by treating them with compounds according to formula (I).

The compounds of formula (I) are known from WO 99/024 413, WO 2006/098128, JP 2007-84566 and WO 96/29871, inter alia.

Preference is given to compounds of the formula (I) in which
A is C-Hal or nitrogen,
B is nitrogen,
R¹ is halogen, C₁-C₆-alkyl, C₂-C₆-alkenyl, C₃-C₆-cycloalkyl,
R² is hydroxyl, C₁-C₆-thioalkyl, C₁-C₆-aminoalkyl, C₁-C₆-alkoxy, aniline, phenoxy,
or is C₁-C₆-alkoxy which is substituted by cyano, halogen, C₁-C₆-alkyl, C₁-C₆-alkoxy, C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl, or
is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is fluorine, chlorine or bromine.

Particular preference is given to compounds according to formula (I) in which Hal is chlorine or bromine.

Particular preference is given to compounds according to formula (I) in which Hal is chlorine.

Particular preference is given to compounds according to formula (I) in which A is C-Hal or nitrogen and B is nitrogen.

Particular preference is given to compounds according to formula (I) in which A is C-Hal and B is nitrogen.

Particular preference is given to compounds according to formula (I) in which A is nitrogen and B is nitrogen.

Particular preference is given to compounds according to formula (I) in which A is C-Hal or nitrogen and B is nitrogen, and R² is aniline, or is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl.

Particular preference is given to compounds according to formula (I) in which A is C-Hal or nitrogen and B is nitrogen, and R² is hydroxyl.

Particular preference is given to compounds according to formula (I) in which A is nitrogen and B is nitrogen, and R² is hydroxyl.

Particular preference is given to compounds according to formula (I) in which A is C-Hal or nitrogen and B is nitrogen, and R² is C₁-C₆-thioalkyl.

Particular preference is given to compounds according to formula (I) in which A is C-Hal or nitrogen and B is nitrogen, and R² is C₁-C₆-alkoxy.

In particular, the compounds according to formula (I) are as shown in Table 1.

| No | A | B | R¹ | R² | Hal |
|---|---|---|---|---|---|
| I-1 | N | N | CH₃ | 3-chloro-4- | — |
| | | | | methylaniline | |
| I-2 | N | N | CH₃ | OH | — |
| I-3 | N | N | CH₃ | methoxy | — |
| I-4 | N | N | CH₃ | thiomethyl | — |
| I-5 | N | N | CH₃ | 2-cyanoaniline | — |
| I-6 | N | N | cyclopropyl | 3-chloro-4- | — |
| | | | | methylaniline | |
| I-7 | N | N | cyclopropyl | OH | — |
| I-8 | N | N | cyclopropyl | methoxy | — |
| I-9 | N | N | cyclopropyl | thiomethyl | — |
| I-10 | N | N | cyclopropyl | 2-cyanoaniline | — |
| I-11 | C-Hal | N | Cl | 3-chloro-4- | Cl |
| | | | | methylaniline | |
| I-12 | C-Hal | N | Cl | OH | Cl |
| I-13 | C-Hal | N | Cl | methoxy | Cl |
| I-14 | C-Hal | N | Cl | thiomethyl | Cl |
| I-15 | C-Hal | N | Cl | 2-cyanoaniline | Cl |
| I-16 | C-Hal | N | CH₃ | 3-chloro-4- | Cl |
| | | | | methylaniline | |
| I-17 | C-Hal | N | CH₃ | OH | Cl |
| I-18 | C-Hal | N | CH₃ | methoxy | Cl |
| I-19 | C-Hal | N | CH₃ | thiomethyl | Cl |
| I-20 | C-Hal | N | CH₃ | 2-cyanoaniline | Cl |

(C₁-C₆)-Alkyl represents a straight-chain or branched alkyl radical having 1 to 6 carbon atoms. Preferred is a straight-chain or branched alkyl radical having 1 to 4, particularly preferably having 1 to 3, carbon atoms. The following may be mentioned by way of example and by preference: methyl, ethyl, n-propyl, isopropyl, tert-butyl, n-pentyl and n-hexyl.

Halogen represents fluorine, chlorine, bromine and iodine. Preference is given to fluorine, chlorine and bromine. Particular preference is given to bromine and chlorine.

(C₃-C₆)-Cycloalkyl represents cyclopropyl, cyclopentyl, cyclobutyl or cyclohexyl. Preference is given to cyclopropyl, cyclopentyl and cyclohexyl. Particular preference is given to cyclopropyl.

(C₂-C₆)-Alkenyl represents a straight-chain or branched alkenyl radical having 2 to 6 carbon atoms. Preferred is a straight-chain or branched alkenyl radical having 2 to 4, particularly preferably having 2 to 3, carbon atoms. The following may be mentioned by way of example and by preference: vinyl, allyl, n-prop-1-en-1-yl and n-but-2-en-1-yl.

(C₁-C₆)-Alkoxy represents a straight-chain or branched alkoxy radical having 1 to 6 carbon atoms. Preferred is a straight-chain or branched alkoxy radical having 1 to 4, particularly preferably having 1 to 3, carbon atoms. The following may be mentioned by way of example and by preference: methoxy, ethoxy, n-propoxy, isopropoxy, tert-butoxy, n-pentoxy and n-hexoxy.

(C₁-C₆)-Alkoxycarbonyl represents a straight-chain or branched alkoxycarbonyl radical having 1 to 6 carbon atoms. Preferred is a straight-chain or branched alkoxycarbonyl radical having 1 to 4, particularly preferably having 1 to 3, carbon atoms. The following may be mentioned by way of example and by preference: methoxycarbonyl, ethoxycarbonyl, n-propoxycarbonyl, isopropoxycarbonyl and tert-butoxycarbonyl.

(C₁-C₆)-Alkylcarbonyl represents a straight-chain or branched alkylcarbonyl radical having 1 to 6 carbon atoms. Preferred is a straight-chain or branched alkylcarbonyl radical having 1 to 4, particularly preferably having 1 to 3, carbon atoms. The following may be mentioned by way of example and by preference: methylcarbonyl, ethylcarbonyl, n-propylcarbonyl, isopropylcarbonyl and tert-butylcarbonyl.

The general or preferred radical definitions given above may be combined as desired between the respective ranges and preferred ranges.

Bacteria as pathogens in useful plants are encountered inter alia in temperate or moist-warm climates, where the cause bacterioses in a large number of useful plants with in some cases considerable economic losses. Infection with Erwinia species, for example, may cause the death of entire fruit plantations such as apples or pears. Also known is bacterial soft rot in potatoes, tumour formation in plants caused by infection with agrobacteria and also a large number of necrotic diseases when cereals such as wheat or rice, vegetables or citrus fruit are infected by Xanthomonas species.

Bacterioses caused by Pseudomonas species in particular in vegetables, tree fruits and tobacco are considered as particularly destructive.

As is to be expected, fungicidally active compounds whose mechanism of action is based on the modulation of fungus-specific metabolic processes are ineffective against bacteria. Accordingly, the use of antibiotics such as streptomycin, blasticidin S or kasugamycin is, in principle, the only effective way for controlling bacteria in useful plants, too. However, this approach is adopted only in rare cases since these antibiotics rely on the same mechanisms of action as antibiotics in human and veterinary medicine, and there are therefore huge reservations against the use of antibiotics in crop protection. There are concerns that the formation of resistance is promoted; moreover, most antibiotics are expensive and can frequently only be obtained by employing biotechnological methods, inter alia.

There is therefore a great need for broadly effective methods for controlling bacterial diseases in useful plants, which methods furthermore require only small amounts of substance to be applied and, in addition, do not damage the plants.

It has now been found that the compounds according to formula (I) are particularly suitable for controlling bacterial harmful organisms in useful plants.

Accordingly, the invention firstly provides the use of a compound selected from the compounds according to formula (I),

where
A is nitrogen or C-Hal,
B is nitrogen or C-Hal,
R¹ is hydrogen, halogen, cyano, C₁-C₆-alkyl, C₂-C₆-alkenyl, C₃-C₆-cycloalkyl, phenyl, C₁-C₆-alkyl which is substituted by Hal or cyano, C₂-C₆-alkenyl which is substituted by Hal or cyano, C₃-C₆-cycloalkyl which is substituted by Hal or cyano, or phenyl which is substituted by cyano, halogen, alkoxy,
R² is hydroxyl, C₁-C₆-thioalkyl, C₁-C₆-aminoalkyl, C₁-C₆-alkoxy, aniline, phenoxy,
or is C₁-C₆-alkoxy which is substituted by cyano, halogen, C₁-C₆-alkyl, C₁-C₆-alkoxy and/or C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl or is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is halogen,
   for controlling bacterial harmful organisms in useful plants.

Accordingly, the invention furthermore provides the use of a compound selected from the compounds according to formula (I) in which
A is C-Hal or nitrogen,
B is nitrogen,
R¹ is halogen, C₁-C₆-alkyl, C₂-C₆-alkenyl, C₃-C₆-cycloalkyl,
R² is hydroxyl, C₁-C₆-thioalkyl, C₁-C₆-aminoalkyl, C₁-C₆-alkoxy, aniline, phenoxy,
or is C₁-C₆-alkoxy which is substituted by cyano, halogen, C₁-C₆-alkyl, C₁-C₆-alkoxy, C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl, or
is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is fluorine, chlorine or bromine,
   for controlling bacterial harmful organisms in useful plants.

Accordingly, the invention furthermore provides the use of a compound selected from the compounds according to formula (I) in which
Hal is chlorine or bromine,
   for controlling bacterial harmful organisms in useful plants.

Accordingly, the invention furthermore provides the use of a compound selected from the compounds according to formula (I) in which
Hal is chlorine,
   for controlling bacterial harmful organisms in useful plants.

Accordingly, the invention furthermore provides the use of a compound selected from the compounds according to formula (I) in which
A is C-Hal or nitrogen and B is nitrogen,
   for controlling bacterial harmful organisms in useful plants.

Accordingly, the invention furthermore provides the use of a compound selected from the compounds according to formula (I) in which
A is nitrogen and B is nitrogen,
   for controlling bacterial harmful organisms in useful plants.

Accordingly, the invention furthermore provides the use of a compound selected from the compounds according to formula (I) in which
A is C-Hal or nitrogen and B nitrogen, and R² is aniline or aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
   for controlling bacterial harmful organisms in useful plants.

Accordingly, the invention furthermore provides the use of a compound selected from the compounds according to formula (I) in which
A is C-Hal or nitrogen and B is nitrogen, and R² is hydroxyl,
   for controlling bacterial harmful organisms in useful plants.

Accordingly, the invention furthermore provides the use of a compound selected from the compounds according to formula (I) in which
A is nitrogen and B is nitrogen, and R² is hydroxyl,
   for controlling bacterial harmful organisms in useful plants.

Accordingly, the invention furthermore provides the use of a compound selected from the compounds according to formula (I) in which
A is C-Hal or nitrogen and B is nitrogen, and R² is C₁-C₆-thioalkyl,
   for controlling bacterial harmful organisms in useful plants.

Accordingly, the invention furthermore provides the use of a compound selected from the compounds according to formula (I) in which
A is -Hal or nitrogen and B is nitrogen, and R² is C₁-C₆-alkoxy,
   for controlling bacterial harmful organisms in useful plants.

Accordingly, the invention furthermore provides the use of compound I-1 for controlling bacterial harmful organisms in useful plants.

Accordingly, the invention furthermore provides the use of compound I-1 for controlling Xanthomonadaceae in useful plants.

Accordingly, the invention furthermore provides the use of compound I-1 for controlling Xanthomonadaceae in rice.

Accordingly, the invention furthermore provides the use of compound I-1 for controlling Xanthomonas oryzae in rice.

Accordingly, the invention furthermore provides the use of compound I-2 for controlling bacterial harmful organisms in useful plants.

Accordingly, the invention furthermore provides the use of compound I-2 for controlling Xanthomonadaceae in useful plants.

Accordingly, the invention furthermore provides the use of compound I-2 for controlling Xanthomonadaceae in rice.

Accordingly, the invention furthermore provides the use of compound I-2 for controlling Xanthomonas oryzae in rice.

Accordingly, the invention furthermore provides the use of compound I-3 for controlling bacterial harmful organisms in useful plants.

Accordingly, the invention furthermore provides the use of compound I-4 for controlling bacterial harmful organisms in useful plants.

Accordingly, the invention furthermore provides the use of compound I-5 for controlling bacterial harmful organisms in useful plants.

Accordingly, the invention furthermore provides the use of compound I-6 for controlling bacterial harmful organisms in useful plants.

Accordingly, the invention furthermore provides the use of compound I-7 for controlling bacterial harmful organisms in useful plants.

Accordingly, the invention furthermore provides the use of compound I-8 for controlling bacterial harmful organisms in useful plants.

Accordingly, the invention furthermore provides the use of compound I-9 for controlling bacterial harmful organisms in useful plants.

Accordingly, the invention furthermore provides the use of compound I-10 for controlling bacterial harmful organisms in useful plants.

Accordingly, the invention furthermore provides the use of compound I-11 for controlling bacterial harmful organisms in useful plants.

Accordingly, the invention furthermore provides the use of compound I-12 for controlling bacterial harmful organisms in useful plants.

Accordingly, the invention furthermore provides the use of compound I-12 for controlling Xanthomonadaceae in useful plants.

Accordingly, the invention furthermore provides the use of compound I-12 for controlling Xanthomonadaceae in rice.

Accordingly, the invention furthermore provides the use of compound I-12 for controlling Xanthomonas oryzae in rice.

Accordingly, the invention furthermore provides the use of compound I-13 for controlling bacterial harmful organisms in useful plants.

Accordingly, the invention furthermore provides the use of compound I-14 for controlling bacterial harmful organisms in useful plants.

Accordingly, the invention furthermore provides the use of compound I-15 for controlling bacterial harmful organisms in useful plants.

Accordingly, the invention furthermore provides the use of compound I-15 for controlling Xanthomonadaceae in useful plants.

Accordingly, the invention furthermore provides the use of compound I-15 for controlling Xanthomonadaceae in rice.

Accordingly, the invention furthermore provides the use of compound I-15 for controlling Xanthomonas oryzae in rice.

Accordingly, the invention furthermore provides the use of compound I-16 for controlling bacterial harmful organisms in useful plants.

Accordingly, the invention furthermore provides the use of compound I-17 for controlling bacterial harmful organisms in useful plants.

Accordingly, the invention furthermore provides the use of compound I-18 for controlling bacterial harmful organisms in useful plants.

Accordingly, the invention furthermore provides the use of compound I-19 for controlling bacterial harmful organisms in useful plants.

Accordingly, the invention furthermore provides the use of compound I-20 for controlling bacterial harmful organisms in useful plants.

The invention furthermore provides a method for controlling bacterial harmful organisms in useful plants, characterized in that the plants are treated with a compound selected from the compounds according to formula (I),

where
A is nitrogen or C-Hal,
B is nitrogen or C-Hal,
R¹ is hydrogen, halogen, cyano, C₁-C₆-alkyl, C₂-C₆-alkenyl, C₃-C₆-cycloalkyl, phenyl, C₁-C₆-alkyl which is substituted by Hal or cyano, C₂-C₆-alkenyl which is substituted by Hal or cyano, C₃-C₆-cycloalkyl which is substituted by Hal or cyano, or phenyl which is substituted by cyano, halogen, alkoxy,
R² is hydroxyl, C₁-C₆-thioalkyl, C₁-C₆-aminoalkyl, C₁-C₆-alkoxy, phenoxy, aniline,
or is C₁-C₆-alkoxy which is substituted by cyano, halogen, C₁-C₆-alkyl, C₁-C₆-alkoxy and/or C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl or is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is halogen

The invention furthermore provides a method for controlling bacterial harmful organisms in useful plants, characterized in that the plants are treated with a compound selected from the compounds according to formula (I) in which
A is C-Hal or nitrogen,
B is nitrogen,
R¹ is halogen, C₁-C₆-alkyl, C₂-C₆-alkenyl, C₃-C₆-cycloalkyl,
R² is hydroxyl, C₁-C₆-thioalkyl, C₁-C₆-aminoalkyl, C₁-C₆-alkoxy, aniline, phenoxy,
or is C₁-C₆-alkoxy which is substituted by cyano, halogen, C₁-C₆-alkyl, C₁-C₆-alkoxy, C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl, or
is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is fluorine, chlorine or bromine.

The invention furthermore provides a method for controlling bacterial harmful organisms in useful plants, characterized in that the plants are treated with a compound selected from the compounds according to formula (I) in which
Hal is chlorine or bromine.

The invention furthermore provides a method for controlling bacterial harmful organisms in useful plants, characterized in that the plants are treated with a compound selected from the compounds according to formula (I) in which
Hal is chlorine.

The invention furthermore provides a method for controlling bacterial harmful organisms in useful plants, characterized in that the plants are treated with a compound selected from the compounds according to formula (I) in which
A is C-Hal or nitrogen and B is nitrogen.

The invention furthermore provides a method for controlling bacterial harmful organisms in useful plants, characterized in that the plants are treated with a compound selected from the compounds according to formula (I) in which
A is nitrogen and B is nitrogen.

The invention furthermore provides a method for controlling bacterial harmful organisms in useful plants, characterized in that the plants are treated with a compound selected from the compounds according to formula (I) in which
A is C-Hal or nitrogen and B is nitrogen, and R² is aniline, or is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl.

The invention furthermore provides a method for controlling bacterial harmful organisms in useful plants, characterized in that the plants are treated with a compound selected from the compounds according to formula (I) in which
A is C-Hal or nitrogen and B is nitrogen, and R² is hydroxyl.

The invention furthermore provides a method for controlling bacterial harmful organisms in useful plants, characterized in that the plants are treated with a compound selected from the compounds according to formula (I) in which
A is nitrogen and B is nitrogen, and R² is hydroxyl.

The invention furthermore provides a method for controlling bacterial harmful organisms in useful plants, characterized in that the plants are treated with a compound selected from the compounds according to formula (I) in which
A is C-Hal or nitrogen and B is nitrogen, and R² is C₁-C₆-thioalkyl.

The invention furthermore provides a method for controlling bacterial harmful organisms in useful plants, characterized in that the plants are treated with a compound selected from the compounds according to formula (I) in which
A is C-Hal or nitrogen and B is nitrogen, and R² is C₁-C₆-alkoxy.

The invention furthermore provides a method for controlling bacterial harmful organisms in useful plants, characterized in that the plants are treated with compound I-1.

The invention furthermore provides a method for controlling Xanthomonadaceae in useful plants, characterized in that the plants are treated with compound I-1.

The invention furthermore provides a method for controlling Xanthomonas oryzae in useful plants, characterized in that the plants are treated with compound I-1.

The invention furthermore provides a method for controlling Xanthomonadaceae in rice, characterized in that the plants are treated with compound I-1.

The invention furthermore provides a method for controlling bacterial harmful organisms in useful plants, characterized in that the plants are treated with compound I-2.

The invention furthermore provides a method for controlling Xanthomonadaceae in useful plants, characterized in that the plants are treated with compound I-2.

The invention furthermore provides a method for controlling Xanthomonas oryzae in useful plants, characterized in that the plants are treated with compound I-2.

The invention furthermore provides a method for controlling Xanthomonadaceae in rice, characterized in that the plants are treated with compound I-2.

The invention furthermore provides a method for controlling bacterial harmful organisms in useful plants, characterized in that the plants are treated with compound I-3.

The invention furthermore provides a method for controlling bacterial harmful organisms in useful plants, characterized in that the plants are treated with compound I-4.

The invention furthermore provides a method for controlling bacterial harmful organisms in useful plants, characterized in that the plants are treated with compound I-5.

The invention furthermore provides a method for controlling bacterial harmful organisms in useful plants, characterized in that the plants are treated with compound I-6.

The invention furthermore provides a method for controlling bacterial harmful organisms in useful plants, characterized in that the plants are treated with compound I-7.

The invention furthermore provides a method for controlling bacterial harmful organisms in useful plants, characterized in that the plants are treated with compound I-8.

The invention furthermore provides a method for controlling bacterial harmful organisms in useful plants, characterized in that the plants are treated with compound I-9.

The invention furthermore provides a method for controlling bacterial harmful organisms in useful plants, characterized in that the plants are treated with compound I-10.

The invention furthermore provides a method for controlling bacterial harmful organisms in useful plants, characterized in that the plants are treated with compound I-11.

The invention furthermore provides a method for controlling bacterial harmful organisms in useful plants, characterized in that the plants are treated with compound I-12.

The invention furthermore provides a method for controlling Xanthomonadaceae in useful plants, characterized in that the plants are treated with compound I-12.

The invention furthermore provides a method for controlling Xanthomonas oryzae in useful plants, characterized in that the plants are treated with compound I-12.

The invention furthermore provides a method for controlling Xanthomonadaceae in rice, characterized in that the plants are treated with compound I-12.

The invention furthermore provides a method for controlling bacterial harmful organisms in useful plants, characterized in that the plants are treated with compound I-13.

The invention furthermore provides a method for controlling bacterial harmful organisms in useful plants, characterized in that the plants are treated with compound I-14.

The invention furthermore provides a method for controlling bacterial harmful organisms in useful plants, characterized in that the plants are treated with compound I-15.

The invention furthermore provides a method for controlling Xanthomonadaceae in useful plants, characterized in that the plants are treated with compound I-15.

The invention furthermore provides a method for controlling Xanthomonas oryzae in useful plants, characterized in that the plants are treated with compound I-15.

The invention furthermore provides a method for controlling Xanthomonadaceae in rice, characterized in that the plants are treated with compound I-15.

The invention furthermore provides a method for controlling bacterial harmful organisms in useful plants, characterized in that the plants are treated with compound I-16.

The invention furthermore provides a method for controlling bacterial harmful organisms in useful plants, characterized in that the plants are treated with compound I-17.

The invention furthermore provides a method for controlling bacterial harmful organisms in useful plants, characterized in that the plants are treated with compound I-18.

The invention furthermore provides a method for controlling bacterial harmful organisms in useful plants, characterized in that the plants are treated with compound I-19.

The invention furthermore provides a method for controlling bacterial harmful organisms in useful plants, characterized in that the plants are treated with compound I-20.

### Definitions

Bacterial harmful organisms include inter alia bacteria causing damage to plants or to a part of a plant.

Bacteria include inter alia Actinobacteria and Proteobacteria.

Of particular interest here are the families of the Xanthomonadaceae, Pseudomonadaceae, Enterobacteriaceae, Microbacteriaceae, Rhizobiaceae.

Examples of phytopathogenic bacteria, in particular in potatoes, cotton, tomato, wheat, barley, rice, soya beans, citrus fruit, etc. are
Acidovorax avena subsp. citrulli
Agrobacterium tumefaciens
Aphelanchoides fragariae
Bacillus subtilis
Clavibacter michiganensis
Clavibacter michiganensis subsp. michiganensis
Clavibacter michiganensis subsp. tessellarius
Clavibacter michiganensis subsp. Sepedonicus
Clavibacter michiganensis subsp. nebraskensis
Clavibacter iranicus
Clavibacter tritici
Corynebacterium fascians
Corynebacterium flaccumfaciens pv. flaccumfaciens
Corynebacterium michiganense
Corynebacterium michiganense pv. tritici
Corynebacterium michiganense pv. nebraskense
Corynebacterium sepedonicum
Curtobacterium flaccumfaciens pv. Flaccumfaciens
Enterobacter dissolvens
Erwinia subspecies
Erwinia ananas
Erwinia carotovora
Erwinia carotovora subsp. atroseptica
Erwinia carotovora subsp. carotovora
Erwinia chrysanthemi
Erwinia chrysanthemi pv. Zeae
Erwinia dissolvens
Erwinia herbicola
Erwinia rhapontic
Erwinia stewartiii
Erwinia tracheiphila
Pantoea agglomerans
Pectobacterium carotovorum
Pectobacterium carotovorum subsp. atrosepticum
Pectobacterium carotovorum subsp. carotovorum
Pectobacterium chrysanthemi
Pseudomonas andropogonis
Pseudomonas avenae subsp. avenae
Pseudomonas corrugata
Pseudomonas fluorescens
Pseudomonas glumae
Pseudomonas fuscovaginae
Pseudomonas marginalis
Pseudomonas marginalis pv. marginalis
Pseudomonas pseudoalcaligenes
Pseudomonas pseudoalcaligenes subsp. citrulli
Pseudomonas solanacearum
Pseudomonas syringae
Pseudomonas syringae pv. atrofaciens
Pseudomonas syringae pv. coronafaciens
Pseudomonas syringae pv. glycinea
Pseudomonas syringae pv. Lachrymans
Pseudomonas syringae pv. maculicola
Pseudomonas syringae pv. striafaciens
Pseudomonas syringae pv. syringae
Pseudomonas syringae pv. Tomato
Pseudomonas syringae pv. tabaci
Ralstonia solanacearum
Rathayibacter tritici
Rhodococcus fascians
Spiroplasma kunkelii
Streptomyces scabiei
Streptomyces scabies
Streptomyces acidiscabies
Streptomyces turgidiscabies
Xanthomonas axonop
Xanthomonas axonopodis pv. glycines
Xanthomonas campestris
Xanthomonas campestris pv. armoraciae
Xanthomonas campestris pv. citrumelo
Xanthomonas campestris pv. citri
Xanthomonas campestris pv. glycines
Xanthomonas campestris pv. Holcicola
Xanthomonas campestris pv. malvacearum
Xanthomonas campestris pv. cucurbitae
Xanthomonas campestris pv. vesicatoria
Xanthomonas campestris pv. translucens
Xanthomonas campestris pv. Oryzae
Xanthomonas fragariae
Xanthomonas oryzae
Xanthomonas oryzae pv. oryzae
Xanthomonas oryzae pv. Oryzicola
Xanthomonas translucens
Xanthomonas translucens pv. Translucens
Xylella fastidiosa

The compound selected from among the compounds according to formula (I) may, if appropriate, be present in the form of mixtures of various isomeric forms which are possible, in particular stereoisomers, such as optical isomers.

The compound selected from among the compounds according to formula (I) can therefore be employed for protecting plants against attack by the abovementioned pathogens within a certain post-treatment period. The period within which protection is afforded generally extends from 1 to 10 days, preferably 1 to 7 days, after the treatment of the plants with the active compounds. Depending on the form of application, the accessibility of the active compounds to the plant can be controlled in a targeted manner.

The good plant tolerance of the compounds according to formula (I) at the concentrations required for controlling plant diseases permits a treatment of aerial and subterranean plant parts, of vegetative propagation material, and of the soil.

The compounds according to formula (I) are also suitable for increasing the yield, show low toxicity and are well tolerated by plants.

In the context of the present invention, on application to plants an advantageous effect was observed.

In accordance with the invention, all plants may be treated. Plants are, in the present context, understood as meaning all plant parts and plant populations, such as desired and undesired wild plants or crop plants (including naturally occurring crop plants). Crop plants may be plants which can be obtained by traditional breeding and optimization methods or else by biotechnological and recombinant methods, or combinations of these methods, including the transgenic plants and including the plant varieties capable or not of being protected by Plant Breeders' Rights. Such methods are, for example, doubled haploids, protoplast fusion, random or targeted mutagenesis and also molecular or genetic markers.

Plant parts are intended to mean all aerial and subterranean parts and organs of the plants, such as herb, pseudostem, shoot, leaf, bract, leaf sheaths, petiole, lamina, flower and root, examples which may be mentioned being leaves, needles, stalks, stems, flowers, fruiting bodies, fruit, banana hand, bunches and seeds, and also roots, tubers, rhizomes, offshoots, suckers, secondary growth. The plant parts also include crop material and vegetative and generative propagation material, for example cuttings, tubers, rhizomes, slips and seeds.

As has already been mentioned above, all plants can be treated in accordance with the invention. In a preferred embodiment, plant species and plant varieties, and their parts, which are found in the wild or which are obtained by conventional biological breeding methods, such as hybridization, meristem cultures, micropropagation, somatic embryogenesis, direct organogenesis or protoplast fusion, are treated. In a further preferred embodiment, transgenic plants and plant varieties which have been obtained by recombinant methods, if appropriate in combination with traditional methods (genetically modified organisms), are treated, such as, for example, transformation by means of Agrobacterium or particle bombardment of embryogenic cells, and micropropagation. Plants include all plant parts as mentioned above.

It is especially preferred to treat, in accordance with the invention, plants of those plant varieties which are in each case commercially available or in use. Plant varieties are understood as meaning plants with new properties (“traits”) which have been obtained by conventional breeding, by mutagenesis or else by recombinant DNA techniques. They may be varieties, breeds, biotypes and genotypes.

Plants which can be treated in accordance with the invention and which may be mentioned are the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana plants and banana plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leeks, onions), Papilionaceae sp. (for example peas); major crop plants such as Gramineae sp. (for example maize, turf, cereals such as wheat, rye, rice, barley, oats, sorghum, millet and triticale), Asteraceae sp. (for example sunflower), Brassicaceae sp. (for example white cabbage, red cabbage, broccoli, cauliflower, Brussels sprouts, pak choi, kohlrabi, small radishes, and also oilseed rape, mustard, horseradish and cress), Fabacae sp. (for example beans, peanuts), Papilionaceae sp. (for example soya beans), Solanaceae sp. (for example potatoes), Chenopodiaceae sp. (for example sugar beet, fodder beet, Swiss chard, beetroot); useful plants and ornamental plants in gardens and forests; and in each case genetically modified types of these plants.

Depending on the plant species or plant varieties, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, better storage stability and/or processability of the harvested products are possible, which exceed the effects which were actually to be expected.

The method of treatment according to the invention can be used in the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example antisense technology, cosuppression technology or RNA interference [RNAi] technology). A heterologous gene that is located in the genome is also called a transgene. A transgene that is defined by its particular location in the plant genome is called a transformation or transgenic event.

Depending on the plant species or plant varieties, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products are possible, which exceed the effects which were actually to be expected

At certain application rates, the compound according to formula (I) may also have a strengthening effect in plants. Accordingly, they are suitable for mobilizing the defence system of the plant against attack by microbial or animal harmful organisms. This may, if appropriate, be one of the reasons for the enhanced activity of the combinations according to the invention, for example against fungi. Plant-strengthening (resistance-inducing) substances are to be understood as meaning, in the present context, also those substances or combinations of substances which are capable of stimulating the defence system of plants in such a way that, when subsequently inoculated with microbial or animal harmful organisms, the treated plants display a substantial degree of resistance to these unwanted phytopathogenic fungi. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant varieties which are preferably to be treated according to the invention include all plants which have genetic material which imparts particularly advantageous, useful traits to these plants (whether obtained by breeding and/or biotechnological means).

Plants and plant varieties which are also preferably to be treated according to the invention are resistant against one or more biotic stresses, i.e. said plants have a better defence against animal and microbial pests, such as against nematodes, insects, mites, phytopathogenic fungi, bacteria, viruses and/or viroids. Musaceae resistant against phytopathogenic fungi or viruses may be mentioned as being preferred here.

Plants and plant varieties which may also be treated according to the invention are those plants which are resistant to one or more abiotic stresses. Abiotic stress conditions may include, for example, drought, cold temperature exposure, heat exposure, osmotic stress, waterlogging, increased soil salinity, increased exposure to minerals, exposure to ozone, exposure to strong light, limited availability of nitrogen nutrients, limited availability of phosphorus nutrients or shade avoidance.

Plants and plant varieties which may also be treated according to the invention are plants in which the vaccines or therapeutic proteins are expressed heterologously. These include, for example, hepatitis B antigen.

Plants and plant varieties which may also be treated according to the invention are those plants characterized by enhanced yield characteristics. Enhanced yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristics of heterosis, or hybrid vigour, which results in generally higher yield, vigour, health and resistance towards biotic and abiotic stress factors. Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally occurring mutations of the abovementioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase are described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD inhibitors can be transformed with a gene encoding a naturally occurring resistant HPPD enzyme, or a gene encoding a mutated HPPD enzyme. Tolerance to HPPD inhibitors can also be obtained by transforming plants with genes encoding certain enzymes enabling the formation of homogentisate despite the inhibition of the native HPPD enzyme by the HPPD inhibitor. Tolerance of plants to HPPD inhibitors can also be improved by transforming plants with a gene encoding an enzyme of prephenate dehydrogenase in addition to a gene encoding an HPPD-tolerant enzyme.

Further herbicide-resistant plants are plants that are made tolerant to acetolactate synthase (ALS) inhibitors. Known ALS inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, selection in cell cultures in the presence of the herbicide or mutation breeding.

Of particular interest is here, as species, rice which has the herbicide tolerances mentioned above.

Of particular interest is the use of the compounds according to the invention on rice comprising the integration event CL121, CL141, CFX51, IMINTA-1, IMINTA-4, LLRICE06, LLRICE62, LLRICE601 and PWC16.

Plants or plant varieties (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

The term “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac.uk/Home/Neil_Crickmore/Bt/, or insecticidal portions thereof, e.g. proteins of the Cry protein classes Cry1Ab, Cry1Ac, Cry1F, Cry2Ab, Cry3Ae, or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, e.g. the Cry1A.105 protein produced by corn event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced into the encoding DNA during cloning or transformation, such as the Cry3Bb1 protein in corn events MON863 or MON88017, or the Cry3A protein in corn event MIR 604;
5) an insecticidal secreted protein from Bacillus thuringiensis or Bacillus cereus, or an insecticidal portion thereof; such as the vegetative insecticidal proteins (VIP) listed at: http://www.lifesci.sussex.ac.uk/Home/Neil_Crickmore/Bt/vip.html, e.g. proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins; or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced into the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant varieties (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARG-encoding genes of the plants or plant cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase.

### Application Forms

The treatment according to the invention of the plants of the Musaceae family and plant parts and of the propagation material with a compound selected from among the compounds according to formula (I) is carried out directly or by acting on their environment, habitat or store by the customary treatment methods, for example by dipping, spraying, atomizing, nebulizing, scattering, painting on, injecting.

In an especially preferred embodiment of the present invention, a compound selected from among the compounds according to formula (I) or their formulations is used for application in the form of granules, for the treatment of vegetative propagation material, or for rhizome or foliar application.

Depending on its respective physical and/or chemical properties, the compound selected from among the compounds according to formula (I) can be converted into the customary formulations, such as solutions, emulsions, suspensions, powders, foams, pastes, granules, sachets, aerosols, microencapsulations in polymeric substances, and ULV cold- and hot-fogging formulations.

These formulations are prepared in a known manner, for example by mixing the compounds according to formula (I) with extenders, that is to say liquid solvents, pressurized liquefied gases and/or solid carriers, optionally with the use of surfactants, that is emulsifiers and/or dispersants and/or foam formers. If water is used as the extender, it is possible for example also to use organic solvents as cosolvents. Liquid solvents which are suitable in the main are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons, such as cyclohexane or paraffins, for example mineral oil fractions, alcohols such as butanol or glycol, and their ethers and esters, ketones such as acetone, methyl ethyl ketone, methyl isobutyl ketone or cyclohexanone, strongly polar solvents such as dimethylformamide and dimethyl sulphoxide, and water, and also mineral, animal and vegetable oils such as, for example, palm oil or other plant seed oils. Liquefied gaseous extenders or carriers are understood as meaning those liquids which are gaseous at normal temperature and under normal pressure, for example aerosol propellants such as halohydrocarbons and butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as highly disperse silica, alumina and silicates. Suitable solid carriers for granules are: for example crushed and fractionated natural rocks such as calcite, pumice, marble, sepiolite, dolomite, and synthetic granules of inorganic and organic meals, and granules of organic material such as sawdust, coconut shells, maize cobs and tobacco stalks. Emulsifiers and/or foam formers which are suitable are: for example nonionic, cationic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, and protein hydrolysates. Suitable dispersants are: for example, lignosulphite waste liquors and methylcellulose.

Adhesives such as carboxymethylcellulose, natural and synthetic polymers in the form of powders, granules or latices, such as gum arabic, polyvinyl alcohol, polyvinyl acetate, and natural phospholipids such as cephalins and lecithins, and synthetic phospholipids, may be used in the formulations. Further additives may be mineral and vegetable oils.

It is possible to use colorants such as inorganic pigments, for example iron oxide, titanium oxide, Prussian Blue, and organic dyestuffs, such as alizarin, azo and metal phthalocyanine dyestuffs, and trace nutrients, such as salts of iron, manganese, boron, copper, cobalt, molybdenum and zinc.

In general, the formulations contain between 0.1 and 95% by weight of active compound, preferably between 0.5 and 90%.

The control of bacterial harmful organisms by treating the vegetative propagation material of plants has been known for a long time and is the subject of continuous improvements. However, the treatment of vegetative propagation material involves a series of problems which cannot always be solved in a satisfactory manner. Thus, it is desirable to develop methods for protecting the vegetative propagation material and the germinating plant which do away with, or at least markedly reduce, the additional application of plant protection products after planting or after emergence of the plants. It is furthermore desirable to optimize the amount of the active compound employed such that the vegetative propagation material and the germinating plant are protected the best possible from attack by bacterial harmful organisms without, however, damaging the plant itself by the active compound employed. In particular, methods for the treatment of vegetative propagation material should also take into consideration the intrinsic properties of transgenic plants in order to achieve an optimal protection of the vegetative propagation material and the germinating plant while keeping the application rate of plant protection products as low as possible.

The present invention therefore relates in particular also to a method of protecting vegetative propagation material and germinating plants from attack by bacterial harmful organisms, by treating the seed and the vegetative propagation material with a composition according to the invention.

The invention also relates to the use of the compositions according to the invention for the treatment of vegetative propagation material for protecting the vegetative propagation material and the germinating plant from bacterial harmful organisms.

One of the advantages of the present invention is that, owing to the special systemic properties of the compositions according to the invention, the treatment of the vegetative propagation material with these compositions protects not only the vegetative propagation material itself, but also the plants which it gives rise to after planting, from bacterial harmful organisms. In this manner, the immediate treatment of the crop at the time of planting, or shortly thereafter, can be dispensed with.

Another advantage is that the compositions according to the invention can be employed in particular also in transgenic vegetative propagation material.

The compositions according to the invention are suitable for protecting vegetative propagation material of any plant variety which is employed in agriculture, in the greenhouse, in forests or in horticulture. In particular, this is vegetative propagation material of cereals such as wheat, barley, rye, oats, maize, rice, triticale and also cotton soya beans, oilseed rape and canola, vegetables such as cucumbers, pumpkins, etc.

Within the scope of the present invention, the composition according to the invention is applied to the vegetative propagation material either alone or in a suitable formulation. Preferably, the vegetative propagation material is treated in a state in which it is sufficiently stable such that no damage occurs during the treatment. In general, the vegetative propagation material can be treated at any point in time between harvesting and planting out. Usually, vegetative propagation material is used which has been separated from the plant and freed from cobs, shells, stalks, coats, hairs or fruit flesh.

When treating the vegetative propagation material, care must be taken in general that the amount of the composition according to the invention, and/or of further additives, applied to the vegetative propagation material is chosen such that the germination of the vegetative propagation material is not adversely affected, or that the plant which it gives rise to is not damaged. This must be considered in particular in the case of active compounds which, at certain application rates, may have phytotoxic effects.

The compositions according to the invention can be applied directly, that is to say without containing further components and without having been diluted. In general, it is preferred to apply the compositions to the vegetative propagation material in the form of a suitable formulation. Suitable formulations and methods for the treatment of seed and of vegetative propagation material are known to the skilled worker.

The compounds which can be used in accordance with the invention and which are selected from among compounds according to formula (I) can be converted into the customary formulations, such as solutions, emulsions, suspensions, powders, foams and ULV formulations.

These formulations are prepared in the known manner by mixing the compounds selected from among the compounds of the formula (I) with customary additives, such as, for example, customary extenders and also solvents or diluents, colorants, wetters, dispersants, emulsifiers, antifoams, preservatives, secondary thickeners, adhesives, gibberellins, mineral and vegetable oils, and also water.

Colorants which may be present in the formulations which can be used in accordance with the invention are all colorants which are customary for such purposes. In this context, both pigments, which are sparingly soluble in water, and dyes, which are soluble in water, may be used. Examples which may be mentioned are the colorants known by the names Rhodamin B, C.I. Pigment Red 112 and C.I. Solvent Red 1.

Wetters which may be present in the formulations which can be used in accordance with the invention are all substances which are customary for formulating agrochemical active compounds and which promote wetting. Alkylnaphthalenesulphonates, such as diisopropyl- or diisobutylnaphthalenesulphonates, may preferably be used.

Suitable dispersants and/or emulsifiers which may be present in the formulations which can be used in accordance with the invention are all nonionic, anionic and cationic dispersants which are conventionally used for the formulation of agrochemical active compounds. The following may be used by preference: nonionic or anionic dispersants or mixtures of nonionic or anionic dispersants. Suitable nonionic dispersants which may be mentioned are, in particular, ethylene oxide/propylene oxide block polymers, alkylphenol polyglycol ethers and tristyrylphenol polyglycol ethers and their phosphated or sulphated derivatives. Suitable anionic dispersants are, in particular, lignosulphonates, salts of polyacrylic acid, and arylsulphonate/formaldehyde condensates.

Antifoams which may be present in the formulations which can be used in accordance with the invention are all foam-inhibitor substances which are conventionally used for the formulation of agrochemical active compounds. Silicone antifoams and magnesium stearate may be used by preference.

Preservatives which may be present in the formulations which can be used in accordance with the invention are all substances which can be employed for such purposes in agrochemical compositions. Examples which may be mentioned are dichlorophene and benzyl alcohol hemiformal.

Secondary thickeners which may be present in the formulations which can be used in accordance with the invention are all substances which can be employed for such purposes in agrochemical compositions. Cellulose derivatives, acrylic acid derivatives, xanthan, modified clays and highly disperse silica are preferably suitable.

Adhesives which may be present in the formulations which can be used in accordance with the invention are all customary binders which can be used in mordants. Polyvinylpyrrolidone, polyvinyl acetate, polyvinyl alcohol and tylose may be mentioned by preference.

Gibberellins which may be present in the formulations which can be used in accordance with the invention are preferably Gibberellin A1, Gibberellin A3 (gibberellic acid), Gibberellin A4, Gibberellin A7. Especially preferred is gibberellic acid.

The gibberellins are known (cf. R. Wegler “Chemie der Pflanzenschutz- and Schädlingsbekämpfungsmittel” [Chemistry of plant protection and pesticide agents], volume 2, Springer Verlag, Berlin-Heidelberg-New York, 1970, pages 401-412).

The formulations which can be used in accordance with the invention can be employed, for the treatment of various types of seed, either directly or after previously having been diluted with water.

Thus, the concentrates or the preparations obtainable therefrom by dilution with water can be employed for dressing the seed. The formulations which can be used in accordance with the invention, or their diluted preparations, can also be employed for treating the vegetative propagation material of transgenic plants. Here, additional synergistic effects may also occur in combination with the substances formed by expression.

The application rate of the formulations which can be used in accordance with the invention can be varied within a substantial range. It depends on the respective active compound content in the formulations, and on the vegetative propagation material. As a rule, the application rates of active compound are between 0.001 and 50 g per kilogram of vegetative propagation material, preferably between 0.01 and 15 g per kilogram of vegetative propagation material.

### Mixtures

A compound selected from among the compounds according to formula (I) can be employed as such or, in formulations, also in a mixture with known fungicides, bactericides, acaricides, nematicides, herbicides, insecticides, safeners, soil-improvement products or products for reducing plant stress, for example Myconate, in order to widen the spectrum of action or to prevent the development of resistance, for example. In many cases, this engenders synergistic effects, that is to say the efficacy of the mixture exceeds the efficacy of the individual components.

In accordance with the invention, the term “mixture” means various combinations of at least two of the abovementioned active compounds which are possible, such as, for example, ready mixes, tank mixes (which is understood as meaning spray slurries prepared from the formulations of the individual active compounds by combining and diluting prior to the application) or combinations of these (for example, a binary ready mix of two of the abovementioned active compounds is made into a tank mix by using a formulation of the third individual substance). According to the invention, the individual active compounds may also be employed sequentially, i.e. one after the other, at a reasonable interval of a few hours or days, in the case of the treatment of seed for example also by applying a plurality of layers which contain different active compounds. Preferably, it is immaterial in which order the individual active compounds can be employed.

The compounds according to formula (I) can be employed as such, in the form of their formulations or the use forms prepared therefrom, such as ready-to-use solutions, suspensions, wettable powders, pastes, soluble powders, dusts and granules. They are applied in the customary manner, for example by pouring, spraying, atomizing, scattering, dusting, foaming, painting on and the like. It is furthermore possible to apply the compounds according to formula (I) by the ultra-low-volume method or to inject the active compound preparation, or the active compound itself, into the soil. The vegetative propagation material of the plants may also be treated.

When employing a compound selected from among the compounds according to formula (I), the application rates may be varied within a substantial range, depending on the type of application. In the treatment of plant parts, the application rates of active compound are generally between 0.1 and 10 000 g/ha, preferably between 10 and 1000 g/ha. In the treatment of vegetative propagation material, the application rates of active compound are generally between 0.001 and 50 g per kilogram of vegetative propagation material, preferably between 0.01 and 10 g per kilogram of vegetative propagation material. In the treatment of the soil, the application rates of active compound are generally between 0.1 and 10 000 g/ha, preferably between 1 and 5000 g/ha.

The good bactericidal activity is illustrated by the example below.

### EXAMPLE

### Xanthomonas campestris pv. Oryzae in Rice

In the Philippines (test station Calauan), a plot test with the rice variety “Mestizo” was initiated to examine the activity of the compound as spray application under agricultural conditions against Xanthomonas campestris pv. Oryzae bacteria.

The products to be tested were treated in the form of sequential spray applications.

The intervals between the individual spray applications varied between 2-3 weeks.

Compound I-15 according to Table 1 was applied in a formulation 200 and compared to an internationally available reference product (product name Shirahagen, active compound techlofthalan (JP-B 56140903), 10 WP) at the application rates stated below in Table 2. The application spray volume was 300 l of water per hectare.

30 days after the second spray application, the control results were evaluated. Scoring was by visual assessment of the degree of infection on leaves in parts of the plot. The efficacy was then calculated using the Abbott formula (Abbott, J. Econ. Entomol. (1925), 18, pp 265-267).

The infection numbers are shown in Table 2 below:

TABLE 2
Effect of compound I-15 against
Xanthomonas campestris pv. Oryzae in rice

| | | Degree of | |
|---|---|---|---|
| | | infection (%) | |
| | Active | (second leaf from | |
| | compound dose | the top - flag leaf) | |
| | (g of active | 30 days after | Efficacy |
| Treatment variant | compound/ha) | the 2nd spraying | (% Abbott) |
| Untreated | | 16 | |
| control | | | |
| Compound I-15 | 200 | 1 | 91 |
| Shirahagen | 200 | 8 | 53 |
| (active compound | | | |
| techlofthalan) | | | |

1. Use of a compound selected from the compounds according to formula (I) 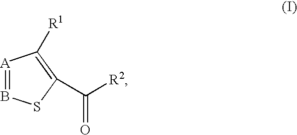
where
A is nitrogen or C-Hal,
B is nitrogen or C-Hal,
R¹ is hydrogen, halogen, cyano, C₁-C₆-alkyl, C₂-C₆-alkenyl, C₃-C₆-cycloalkyl, phenyl, C₁-C₆-alkyl which is substituted by Hal or cyano, C₂-C₆-alkenyl which is substituted by Hal or cyano, C₃-C₆-cycloalkyl which is substituted by Hal or cyano, or phenyl which is substituted by cyano, halogen, alkoxy,
R² is hydroxyl, C₁-C₆-thioalkyl, C₁-C₆-aminoalkyl, C₁-C₆-alkoxy, aniline, phenoxy,
or is C₁-C₆-alkoxy which is substituted by cyano, halogen, C₁-C₆-alkyl, C₁-C₆-alkoxy and/or C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl or is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is halogen,
for controlling bacterial harmful organisms in useful plants.

2. Use of a compound selected from the compounds according to formula (I) where
A is C-Hal or nitrogen,
B is nitrogen,
R¹ is halogen, C₁-C₆-alkyl, C₂-C₆-alkenyl, C₃-C₆-cycloalkyl,
R² is hydroxyl, C₁-C₆-thioalkyl, C₁-C₆-aminoalkyl, C₁-C₆-alkoxy, aniline, phenoxy,
or is C₁-C₆-alkoxy which is substituted by cyano, halogen, C₁-C₆-alkyl, C₁-C₆-alkoxy, C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl, or
is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is fluorine, chlorine or bromine,
for controlling bacterial harmful organisms in useful plants.

3. Use of the compound I-1 according to Table 1 for controlling bacterial harmful organisms in useful plants.

4. Use of the compound I-2 according to Table 1 for controlling bacterial harmful organisms in useful plants.

5. Use of the compound I-12 according to Table 1 for controlling bacterial harmful organisms in useful plants.

6. Use of the compound I-15 according to Table 1 for controlling bacterial harmful organisms in useful plants.

7. Method for controlling bacterial harmful organisms in useful plants, characterized in that the plants are treated with a compound selected from the compounds according to formula (I) 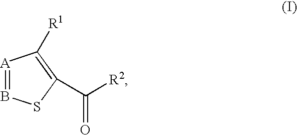
where
A is nitrogen or C-Hal,
B is nitrogen or C-Hal,
R¹ is hydrogen, halogen, cyano, C₁-C₆-alkyl, C₂-C₆-alkenyl, C₃-C₆-cycloalkyl, phenyl, C₁-C₆-alkyl which is substituted by Hal or cyano, C₂-C₆-alkenyl which is substituted by Hal or cyano, C₃-C₆-cycloalkyl which is substituted by Hal or cyano, or phenyl which is substituted by cyano, halogen, alkoxy,
R² is hydroxyl, C₁-C₆-thioalkyl, C₁-C₆-aminoalkyl, C₁-C₆-alkoxy, aniline, phenoxy,
or is C₁-C₆-alkoxy which is substituted by cyano, halogen, C₁-C₆-alkyl, C₁-C₆-alkoxy and/or C₁-C₆-alkylcarbonyl,
or is phenoxy which is substituted by cyano, halogen, C₁-C₆-alkoxy, C₁-C₆-alkylamino, C₁-C₆-alkylcarbonyl, formyl, C₁-C₆-alkyl, C₁-C₆-alkoxy, C₁-C₆-alkylcarbonyl and/or C₁-C₆-alkoxycarbonyl or is aniline which is substituted by cyano, halogen, C₁-C₆-alkyl, C₁-C₆-alkoxy, C₁-C₆-alkylamino, C₁-C₆-dialkylamino and/or C₁-C₆-alkylcarbonyl,
Hal is halogen.

8. Method for controlling bacterial harmful organisms in useful plants, characterized in that the plants are treated with compound I-15 according to Table 1.

9. Method according to claim 8 where the treated plants are transgenic plants.
